(19) United States
(12) Patent Application Publication
(10) Pub. No.: US 2025/0017672 A1
(43) Pub. Date: Jan. 16, 2025

(54) APPLICATION OF TORQUE LIMITS TO SURGICAL ROBOTS

(71) Applicant: Auris Health, Inc., Redwood City, CA (US)

(72) Inventors: Mikael JORDA, Mérignac (FR); Nicholas CHEUNG, Redwood City, CA (US)

(21) Appl. No.: 18/746,443

(22) Filed: Jun. 18, 2024

Related U.S. Application Data
(60) Provisional application No. 63/513,189, filed on Jul. 12, 2023.

(51) Int. Cl. A61B 34/32; A61B 34/10; A61B 90/00; A61B 90/50

(52) U.S. Cl. CPC A61B 34/32; A61B 90/50; A61B 2034/102; A61B 2090/031

(57) ABSTRACT

A surgical robot includes an end effector driven by a plurality of joints located along a robotic arm of the surgical robot. Additionally, the surgical robot includes a processor communicatively coupled to the robotic arm, the processor configured to apply a first torque limit to at least one of the plurality of joints when the robotic arm is actively moving, and apply a second torque limit to at least one of the plurality of joints when the robotic arm is in a stationary state, wherein the second torque limit is different from the first torque limit.

### PRIORITY

This application claims priority to U.S. Provisional Pat. App. No. 63/513,189, entitled “APPLICATION OF TORQUE LIMITS TO SURGICAL ROBOTS,” filed Jul. 12, 2023, the disclosure of which is incorporated by reference herein, in its entirety.

### TECHNICAL FIELD

The systems and methods disclosed herein are directed to devices and methods for indicating locations or orientations of surgical tools, and more particularly to surgical robotic systems for indicating locations or orientations of surgical tools.

### BACKGROUND

A robotically enabled medical system is capable of performing a variety of medical procedures, including both minimally invasive procedures, such as laparoscopy, and non-invasive procedures, such as endoscopy (e.g., bronchoscopy, ureteroscopy, gastroscopy, etc.). In at least some instances, such robotic medical systems may include robotic arms configured to control the movement of end effectors in the form of surgical tool(s) during a given medical procedure. In order to achieve a desired pose of a surgical tool, a robotic arm may be placed into a particular pose during teleoperation. In addition, some robotically enabled medical systems may include an arm support that is connected to respective bases of the robotic arms and supports the robotic arms.

### SUMMARY

An embodiment of a surgical robot comprises an end effector driven by a plurality of joints located along a robotic arm of the surgical robot, and a processor communicatively coupled to the robotic arm, the processor configured to apply a first torque limit to at least one of the plurality of joints when the robotic arm is actively moving, and apply a second torque limit to at least one of the plurality of joints when the robotic arm is in a stationary state, wherein the second torque limit is different from the first torque limit. In some embodiments, the processor is configured to determine the first torque limit using a dynamic model of the robotic arm. In some embodiments, the processor is configured to estimate an expected torque producible by the plurality of joints to satisfy a control input applied to the robotic arm, wherein the expected torque is based on a predefined dynamic model of the robotic arm. In certain embodiments, the expected torque comprises a sum of a plurality of torques required to overcome gravitational loads applied to the robotic arm, frictional loads applied to one or more of the plurality of joints, and dynamic loads resulting from movement of one or more joints of the robotic arm. In certain embodiments, the expected torque includes an estimate of an error of the predefined dynamic model upon which the expected torque is based. In some embodiments, the expected torque comprises an estimated expected tissue load applied to the robotic arm during the performance of a clinical task. In some embodiments, the expected torque comprises an initial expected torque and the processor is configured to determine a final expected torque range that is bounded between an upper bound and a lower bound that is less than the upper bound. In certain embodiments, the first torque limit corresponds to a maximum permissible force that the robotic arm is able to apply to an object external to the robotic arm. In certain embodiments, the processor is configured to determine when the robotic arm is actively moving, and to determine when the robotic arm is in the stationary state. In some embodiments, the second torque limit is greater than the first torque limit.

An embodiment of a surgical robot comprises an end effector driven by a plurality of joints of a robotic arm of the surgical robot, and a processor communicatively coupled to the robotic arm, the processor configured to estimate an expected torque producible by the plurality of joints to satisfy a control input applied to the robotic arm, wherein the expected torque is based on a predefined dynamic model of the robotic arm, and apply a torque limit to at least one of the plurality of joints that is based on and greater than the expected torque. In some embodiments, the expected torque comprises a sum of a plurality of torques required to overcome gravitational loads applied to the robotic arm, frictional loads applied to the plurality of joints, and dynamic loads resulting from movement of one or more joints of the robotic arm. In certain embodiments, the expected torque includes an estimate of a model error caused by the predefined dynamic model upon which the expected torque is based. In certain embodiments, the expected torque includes an external interference parameter corresponding to an estimated drag applied to the robotic arm by objects external to the robotic arm. In some embodiments, the expected torque comprises an initial expected torque, and wherein the processor is configured to determine a final expected torque that is bounded between an upper bound and a lower bound that is less than the upper bound. In some embodiments, the upper bound comprises a sum of the initial expected torque and an external interference parameter corresponding to an estimated drag applied to the robotic arm by objects external to the robotic arm, and the lower bound comprises a difference between the initial expected torque and the external interference parameter. In certain embodiments, the upper bound comprises a sum of the initial expected torque, an external interference parameter corresponding to an estimated drag applied to the robotic arm by objects external to the robotic arm, and an estimate of a model error caused by the predefined dynamic model upon which the expected torque is based, and the lower bound comprises a difference between the initial expected torque and the sum of the external interference parameter and the model error.

An embodiment of a surgical robot comprises a first end effector driven by a plurality of first joints of a first robotic arm of the surgical robot, a second end effector driven by a plurality of second joints of a second robotic arm of the surgical robot, and a processor communicatively coupled to the first robotic arm and the second robotic arm, the processor configured to apply a first torque limit to at least one of the plurality of first joints and/or at least one of the plurality of second joints in response to determining by the processor that the first robotic arm and/or the second robotic arm is actively moving, and apply a second torque limit to at least one of the plurality of first joints and/or at least one of the plurality of second joints in response to determining by the processor that the first robotic arm and/or the second robotic arm is in a stationary state, wherein the second torque limit is different from the first torque limit. In certain embodiments, the first torque limit corresponds to a maximum permissible force that may be applied by either the first robotic arm and the second robotic arm to an object external the respective first and second robotic arms. In some embodiments, the processor is configured to determine the first torque limit using a dynamic model of at least one of the first robotic arm and the second robotic arm. In some embodiments, the first robotic arm and the second robotic arm are held stationary with respect to a support structure physically supporting the first robotic arm and the second robotic arm when in the stationary state. In certain embodiments, the second torque limit is greater than the first torque limit.

The various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, the language used in the specification has been principally selected for readability and instructional purposes and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an exemplary robotic medical system according to some embodiments.

FIG. 2 illustrates another view of an exemplary robotic medical system according to some embodiments.

FIGS. 3A and 3B illustrate different views of an exemplary robotic medical system according to some embodiments.

FIG. 4 illustrates an exemplary view of a robotic medical system with the robotic arms in a stowed position, in accordance with some embodiments.

FIG. 5 illustrates components of a robotic medical system in accordance with some embodiments.

FIGS. 6A to 6C illustrate different views of an exemplary robotic arm according to some embodiments.

FIGS. 7A and 7B illustrate different views of an exemplary robotic arm according to some embodiments.

FIG. 8 illustrates a part of a robotic arm and a surgical tool according to some embodiments.

FIG. 9 illustrates a perspective view of a robotic medical system in accordance with some embodiments.

FIGS. 10-13 illustrate components of another exemplary robotic medical system according to some embodiments.

FIG. 14 is a schematic diagram illustrating electronic components of a robotic medical system in accordance with some embodiments.

### DETAILED DESCRIPTION

### 1. Overview

Aspects of the present disclosure may be integrated into a robotically enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the physician. Additionally, the system may provide the physician with the ability to perform the procedure from an ergonomic position without the need for awkward arm motions and positions. Still further, the system may provide the physician with the ability to perform the procedure with improved case of use such that one or more of the instruments of the system can be controlled by a single user.

Various embodiments will be described below in conjunction with the drawings for purposes of illustration. It should be appreciated that many other embodiments of the disclosed concepts are possible, and various advantages can be achieved with the disclosed embodiments. Headings are included herein for reference and to aid in locating various sections. These headings are not intended to limit the scope of the concepts described with respect thereto. Such concepts may have applicability throughout the entire specification.

### 2. Limiting of Contact Force Applied by a Robotic Arm of a Surgical Robot

This application discloses systems and methods for selectably limiting the contact force applied by a robotic arm of a surgical robot. As will be discussed further herein, during the performance of a teleoperation facilitated by a surgical robot, the robotic arms (along with end effectors coupled thereto) may apply contact forces to one or more external objects including, for example, the patient's body, a user of the surgical robot, and/or other components of the surgical robot (e.g., other robotic arms of the surgical robot). These contact forces may be an expected and routine part of performing a clinical task (e.g., gripping tissues of the patient's body), or they may be unexpected such as a collision between the respective robotic arm and another robotic arm of the surgical robot.

The application of excessive contact forces by the robotic arm may pose a safety risk to the patient and the clinicians operating the surgical robot. For example, excessive contact force applied to the patient's body could potentially injure the patient. Additionally, a collision between an actively moving robotic arm with a stationary robotic arm may result in undesired motion of the formerly stationary robotic arm. For instance, a stationary robotic arm may be employed to statically grip selected tissue of the patient where inadvertent movement of the stationary robotic arm may result in damage to the gripped tissue. Further, the collision between the actively moving robotic arm and the stationary robotic arm may result in damage to one of or both of the robotic arms, or other components of the surgical robot.

The exemplary systems and methods for selectably limiting contact force applied by a robotic arm of a surgical robot address the safety risks posed by contact forces applied by a robotic arm to an external object, whether the external object is the patient's body, a user of the surgical robot, and/or another object such as another robotic arm of the surgical robot. Particularly, in some embodiments, a processor of a surgical robot applies a first torque limit to one or more joints of a robotic arm when the robotic arm is actively moving, and applies a second torque limit to one or more of the joints of the robotic arm when the robotic arm is in a stationary state, wherein the second torque limit is greater than the first torque limit. As will be discussed further herein, by applying a greater torque limit to the stationary robotic arm than that applied to the moving robotic arm, the stationary robotic arm successfully resists moving in response to contact (e.g., a collision) between the moving robotic arm and the stationary robotic arm.

The processor may determine (e.g., continuously, at predefined temporal increments) when each of a plurality of robotic arms of a surgical robot are actively moving and when each is in a stationary state. In some embodiments, the processor of the surgical robot where the first torque limit is applied to robotic arms that are actively moving is based on an expected torque applied by one or more joints of a robotic arm necessary for satisfying a control input applied to the robotic arm as estimated by the processor. For example, the processor may estimate the expected torque based on a dynamic model of the robotic arm whereby the processor may estimate the required torque necessary for executing the control input applied to the robotic arm. In this manner a user of the surgical robot may move a given robotic arm as needed to perform clinical tasks without applying contact forces from the robotic arm to external objects that exceed relevant safety standards.

### A. Robotic System

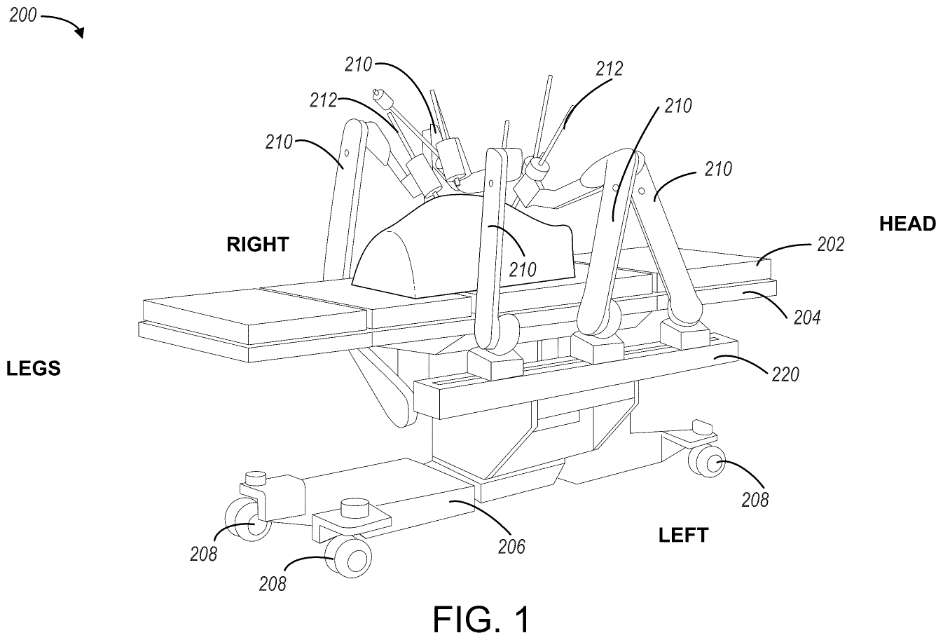

Referring now to FIG. 1, an exemplary robotic surgical or medical system 200 according to some embodiments is illustrated. In some embodiments, the robotic medical system 200 is a robotic surgical system. In this exemplary embodiment, the robotic medical system 200 comprises a patient platform 202 (e.g., a patient platform, a table, a bed) including a support 204 (e.g., a rigid frame). The two ends along the longitudinal length of the patient platform 202 are respectively referred to herein as “head” and “legs”. Additionally, the lateral two sides of the patient platform 202 are respectively referred to herein as “left” and “right.”

In addition to patient platform 202, in this exemplary embodiment, robotic medical system 200 also includes a base 206 for supporting the robotic medical system 200. The base 206 includes wheels 208 that allow the robotic medical system 200 to be easily movable or repositionable in a physical environment. In some embodiments, the wheels 208 are retractable, may be replaced with feet, or may be entirely omitted from the robotic medical system 200 with the base 206 resting directly on the ground.

The robotic medical system 200 includes one or more robotic arms 210. In some embodiments, the robotic arms 210 can be configured to perform robotic medical procedures including, for example, minimally invasive procedures such as laparoscopy. Additionally, although FIG. 1 illustrates five robotic arms 210, it should be appreciated that the robotic medical system 200 may include any number of robotic arms 210, including less than five or six or more.

The robotic medical system 200 also includes one or more support rails 220 (e.g., adjustable arm support or an adjustable bar) that support the robotic arms 210. Each of the robotic arms 210 is supported on, and movably coupled to, a support rail 220, by a respective base joint or actuator of the robotic arm 210. The term “joint” as used herein with respect to robotic arms refers to joints comprising an actuator or motor for powering or driving rotation of the respective joint. In some embodiments, support rail 220 can provide several degrees of freedom (DoFs), including lift, lateral translation, tilt, etc. Each of the robotic arms 210 and/or the support rails 220 may also be referred to as a respective kinematic chain. Additionally, in this exemplary embodiment and as illustrated in FIG. 1, three robotic arms 210 are supported by the support rail 220 that is in the field of view of the figure which is located along the left side of the patient support platform. The two remaining robotic arms 210 are supported by another support rail 220 located along an opposing lateral side of the patient platform 202.

In some embodiments, support rails 220 provide a base position for one or more of the robotic arms 210 for a robotic medical procedure. A robotic arm 210 can be positioned relative to the patient platform 202 by translating the robotic arm 210 along a length of its underlying support rail 220 and/or by adjusting a position and/or orientation of the robotic arm 210 via one or more joints and/or links. In some embodiments, the pose of a given support rail 220 can be changed via manual manipulation, teleoperation, and/or power assisted motion. For example, in some embodiments, the support rail 220 can be translated along a length of the patient platform 202. In certain embodiments, translation of the support rail 220 along a length of the patient platform 202 causes one or more of the robotic arms 210 supported by the support rail 220 to be simultaneously translated with the respective support rail 220 or relative to the support rail 220. In certain embodiments, the support rail 220 can be translated while keeping one or more of the robotic arms stationary with respect to the base 206 of the robotic medical system 200. Additionally, in this exemplary embodiment, the support rail 220 is located along a length of the patient platform 202. In some embodiments, the support rail 220 may extend across a partial or full length of the patient platform 202, and/or across a partial or full width of the patient platform 202.

During a robotic medical procedure, one or more of the robotic arms 210 can also be configured to hold end effectors (e.g., robotically controlled medical instruments or tools, such as an endoscope and/or any other instruments such as sensors, illumination instrument, cutting instrument, etc. that may be used during surgery), and/or be coupled to one or more accessories, including one or more cannulas, in accordance with some embodiments.

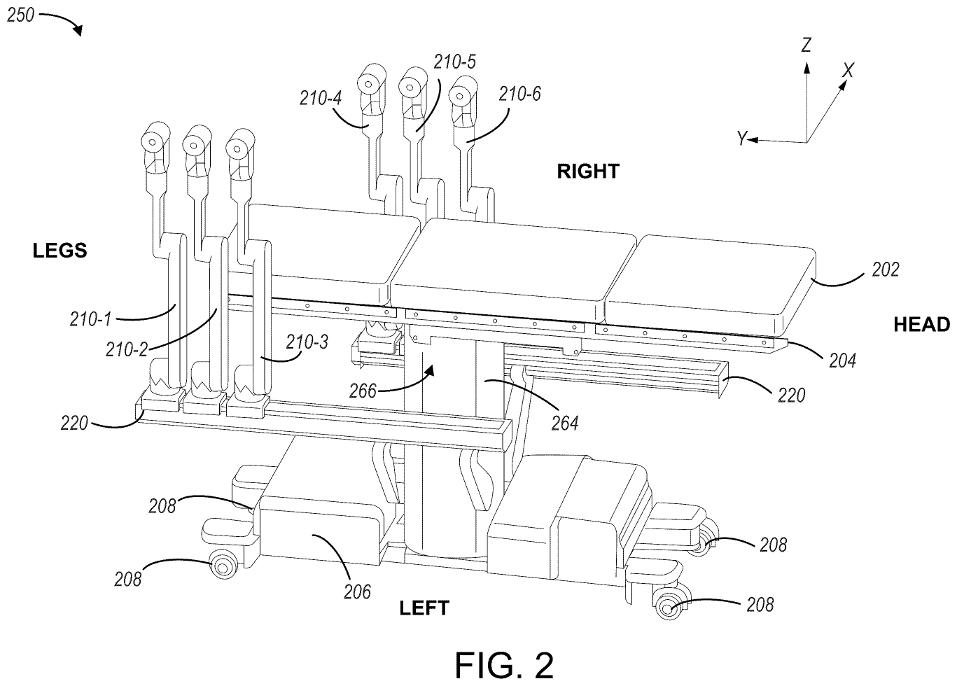

Referring to FIG. 2, another exemplary robotic medical system 250 is shown according to some embodiments. Initially, the robotic medical system 250 includes features in common with the robotic medical system 200 illustrated in FIG. 1, and shared features are labeled similarly. In this exemplary embodiment, the robotic medical system 250 includes six robotic arms indicated in FIGS. 2 as 210-1, 210-2, 210-3, 210-4, 210-5, and 210-6. Additionally, the patient platform 202 is supported by a column 264 that extends between the base 206 and the patient platform 202. In this exemplary embodiment, the patient platform 202 comprises a tilt mechanism 266 positioned between the column 264 and the patient platform 202 to allow the patient platform 202 to pivot, rotate, or tilt relative to the column 264. The tilt mechanism 266 can be configured to allow for lateral and/or longitudinal tilt of the patient platform 202. In some embodiments, the tilt mechanism 266 allows for simultaneous lateral and longitudinal tilt of the patient platform 202.

FIG. 2 illustrates the patient platform 202 in an untilted position. In some embodiments, the untilted position is a default position of the patient platform 202. In some embodiments, the default position of the patient platform 202 is a substantially horizontal position as shown in FIG. 2. As illustrated, in the untilted position, the patient platform 202 can be positioned horizontally or parallel to a surface that supports the robotic medical system 250 (e.g., the ground or floor). In some embodiments, the term “untilted” refers to a state in which the angle between the default position and the current position is less than a threshold angle (e.g., less than 5 degrees, or less than an angle that would cause the patient to shift on the patient platform, etc.). In some embodiments, the term “untilted” refers to a state in which the patient platform is substantially perpendicular to the direction of gravity, irrespective of the angle formed by the surface that supports the robotic medical system relative to gravity.

FIG. 2 illustrates the robotic arms 210-1, 210-2, 210-3, 210-4, 210-5, and 210-6 and the support rails 220 in an exemplary deployed configuration in which the robotic arms 210-1, 210-2, 210-3, 210-4, 210-5, and 210-6 reach above the patient platform 202. In some embodiments, due to the configuration of the robotic medical system 250, which enables stowage of different components beneath the patient platform 202, the robotic arms 210-1, 210-2, 210-3, 210-4, 210-5, and 210-6 and the support rails 220 can occupy a space underneath the patient platform 202. Thus, in some embodiments, the tilt mechanism 266 has a low-profile and/or low volume in order to increase the space available for storage below.

FIG. 2 also illustrates an exemplary, x, y, and z coordinate system that may be used to describe certain features of the embodiments disclosed herein. It will be appreciated that this coordinate system is provided for purposes of example and explanation only and that other coordinate systems may be used. In this exemplary embodiment, the x-direction extends in a lateral direction across the patient platform 202 when the patient platform 202 is in an untilted position. In some configurations, the x-direction extends across the patient platform 202 from one lateral side (e.g., the right side) to the other lateral side (e.g., the left side) when the patient platform 202 is in an untilted position. The y-direction extends in a longitudinal direction along the patient platform 202 when the patient platform 202 is in an untilted position. That is, the y-direction extends along the patient platform 202 from one longitudinal end (e.g., the head end) to the other longitudinal end (e.g., the legs end) when the patient platform 202 is in an untilted position. Additionally, in the untilted position, the patient platform 202 can lie in or be parallel to the x-y plane, which can be parallel to the ground. The z-direction extends along the column 264 in a vertical direction. In some embodiments, the tilt mechanism 266 is configured to laterally tilt the patient platform 202 by rotating the patient platform 202 about a lateral tilt axis that is parallel to the y-axis. The tilt mechanism 266 can further be configured to longitudinally tilt the patient platform 202 by rotating the patient platform 202 about a longitudinal tilt axis that is parallel to the x-axis.

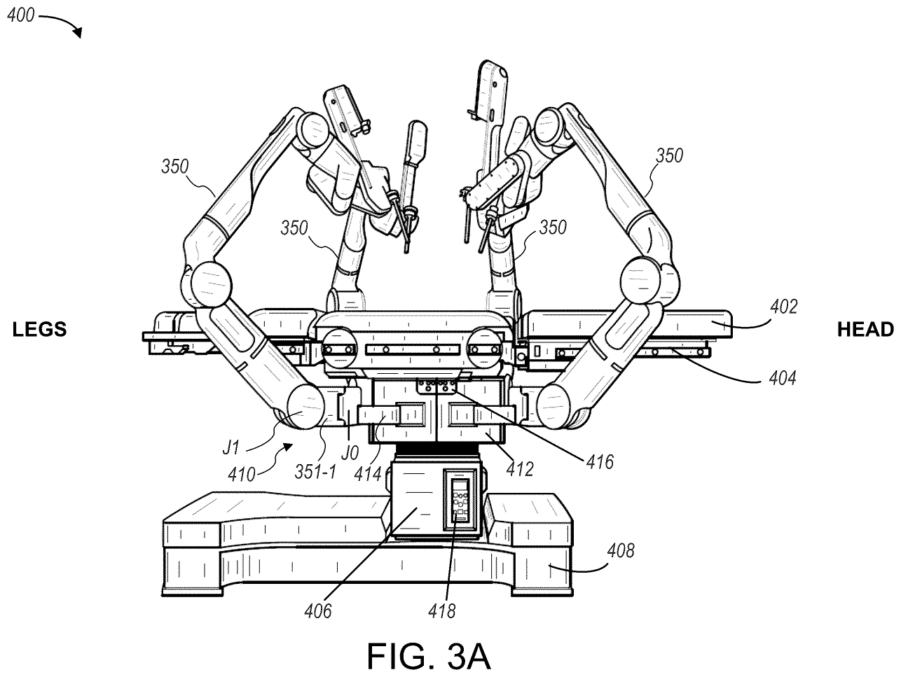

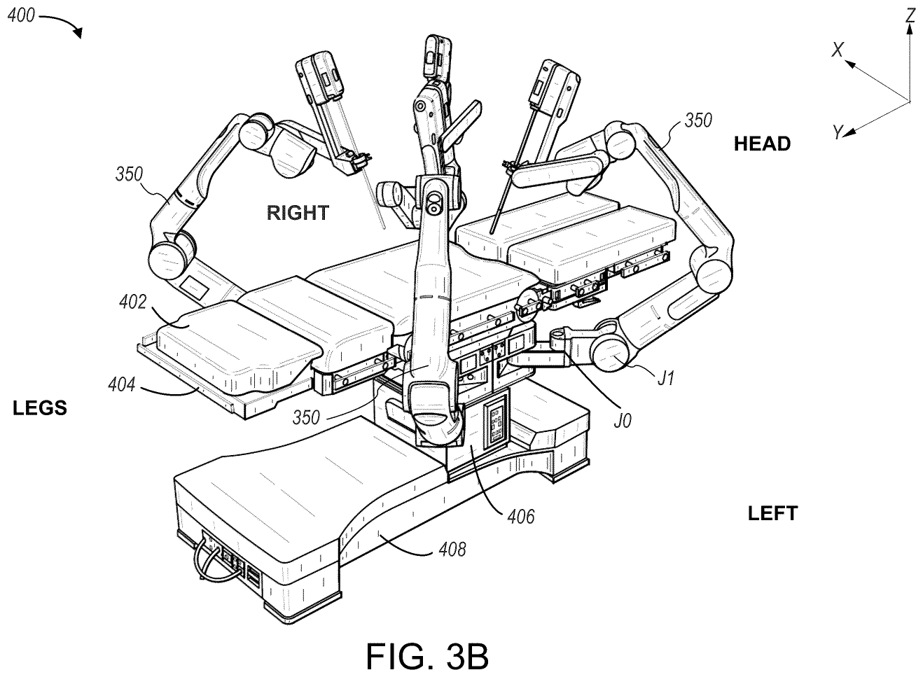

Referring to FIGS. 3A, 3B and 4, another exemplary robotic surgical or medical system 400 is shown according to some embodiments. In this exemplary embodiment, robotic medical system 400 includes a surgical table having a table top 402 on which a patient may be positioned, and which is supported by a table adapter 404. Additionally, robotic medical system 400 includes a support 406 (e.g., a support mechanism, a table column or a pedestal) and a base 408. The support 406 may be mounted to the base 408, which can be fixed to the ground (e.g., a floor of an operating room), or can be movable relative to the ground, e.g., by use of wheels on the base 408. In some embodiments, the various sections of the table top 402 can move relative to each other (e.g., can be tilted or angled relative to each other) and/or the table top 402 can be moved (e.g., tilted, angled) relative to the support 406 and/or the base 408 of the surgical table.

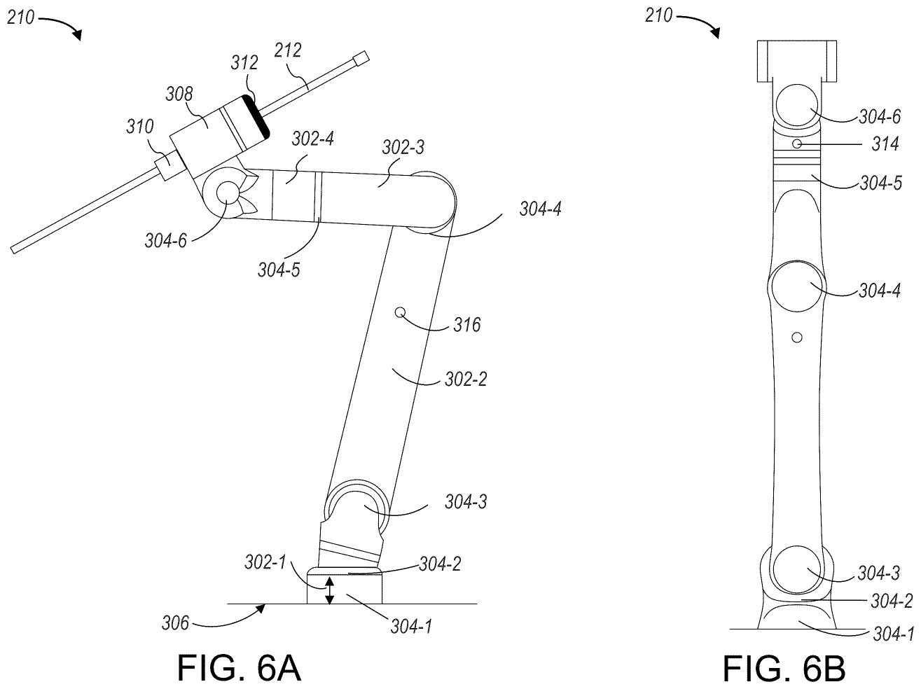

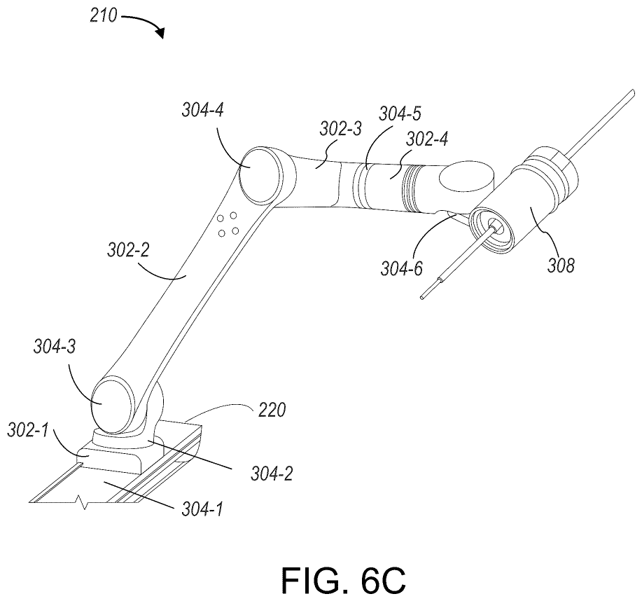

In some embodiments, the robotic medical system 400 includes robotic arms 350 coupled, or couplable to, the surgical table. The robotic arms 350 can be moved between multiple different positions relative to the surgical table, such as, for example, an operating position, a parked position, or a stowed position (e.g., as illustrated in FIG. 4). A given robotic arm 350 can support an end effector such as a medical instrument or tool including, for example, a surgical instrument, tool driver, and/or imaging device. Further details of the robotic arms 350 are described in FIGS. 7A, 7B, and 8 herein. In certain embodiments, the robotic medical system 400 includes one or more input devices (e.g., buttons, switches, touch-sensitive surfaces, etc.), such as a pivot and stow keypad 416 and a table keypad 418.

In some embodiments, a proximal portion of each robotic arm 350 can be implemented as an adapter 410, which may be fixedly coupled to the surgical table. The adapter 410 can include an interface mechanism 412 (e.g., table interface structure), a first link member (e.g., link 414) pivotally coupled to the interface mechanism 412 at a first joint (e.g., joint J0), and coupled to a second link member (e.g., link 351-1) at a second joint (e.g., joint J1). In some embodiments, the second link member 351-1 can be pivotally coupled to the first link member 414 at the second joint J1. In some embodiments, the second link member 351-1 is slidably coupled to the first link member 414 at the second joint J1. The second link member 351-1 is also configured to be coupled to a robotic arm 350 at a coupling that includes a coupling portion of the second link member 351-1 and a coupling portion at a proximal or mounting end portion of the robotic arm 350. The robotic arm 350 also includes a target joint at the mounting end portion of the robotic arm 350. In some embodiments, the target joint is included with the coupling portion at the mounting end portion of the robotic arm 350.

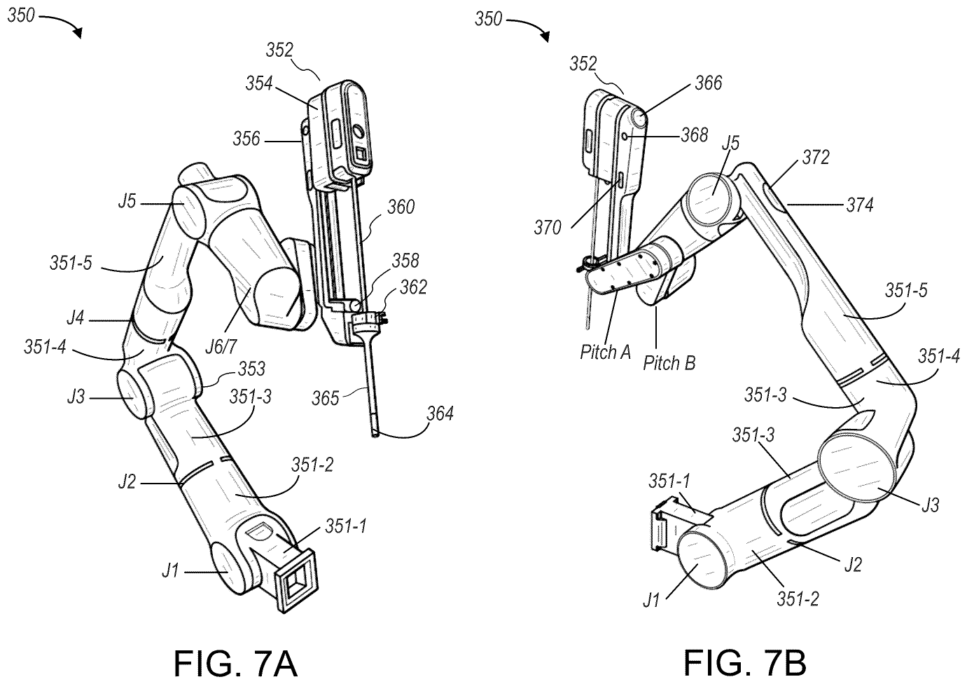

In some embodiments, the distinction between an adapter 410 and robotic arm 350 can be disregarded, and the connection between the surgical table and a distal end of the robotic arm 350 can be conceptualized and implemented as a series of links and joints that provide the desired DoFs for movement of the medical instrument. Details of the links and joints of the robotic arm 350 are described with respect to FIGS. 7A, 7B, and 9 herein.

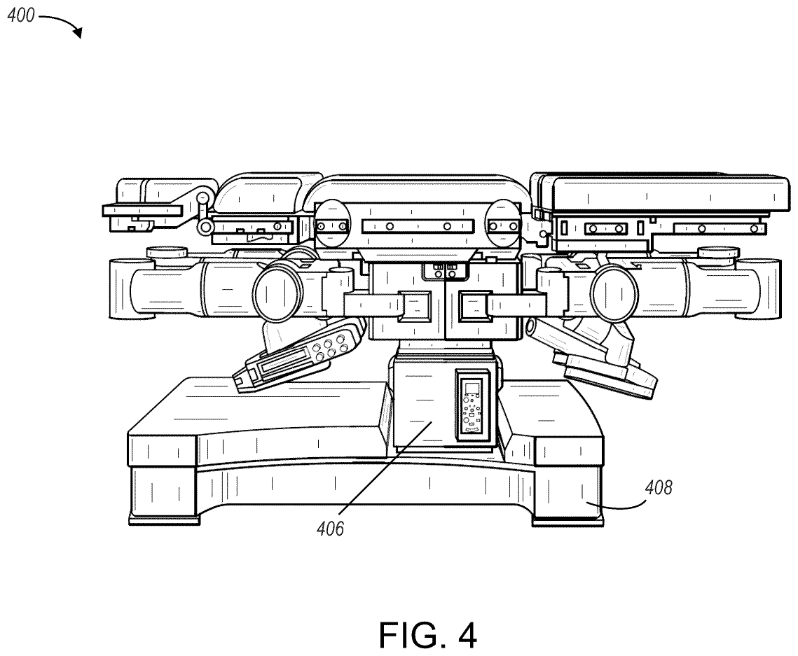

Referring to FIG. 5, in some embodiments, the robotic medical systems described herein (e.g., robotic medical systems 200, 250 and/or 400) includes a tower 230 (e.g., tower viewer) and/or a physician console 240 (or both). The tower 230 may provide support for controls, electronics, fluidics, optics, sensors, and/or power for the patient platform 202 and the physician console 240. In some embodiments, the tower 230 includes a display device 232 including a user interface for displaying a surgical view obtained by one or more cameras of the robotic medical system and/or one or more notifications to an operator of the robotic medical system. In some embodiments, the physician console 240 includes a display device 242 having a user interface usable by a physician operator for operating the patient platform 202. For example, the display device 242 may include a user interface for displaying surgical views obtained by one or more cameras of the robotic medical system and/or one or more notifications to an operator of the robotic medical system. The physician console 240 can provide both robotic controls and pre-operative and real-time information of a medical procedure to a physician operator. In some embodiments, the physician console 240 includes one or more input devices (e.g., buttons, switches, touch-sensitive surfaces, gimbals, etc.), such as, for example, a foot pedal 244. Additionally, in some embodiments, the physician console 240 includes one or more haptic interface devices (HIDs) that provide force and tactile feedback to a user as the user interacts with the physician console 240.

### B. Robotic Arm

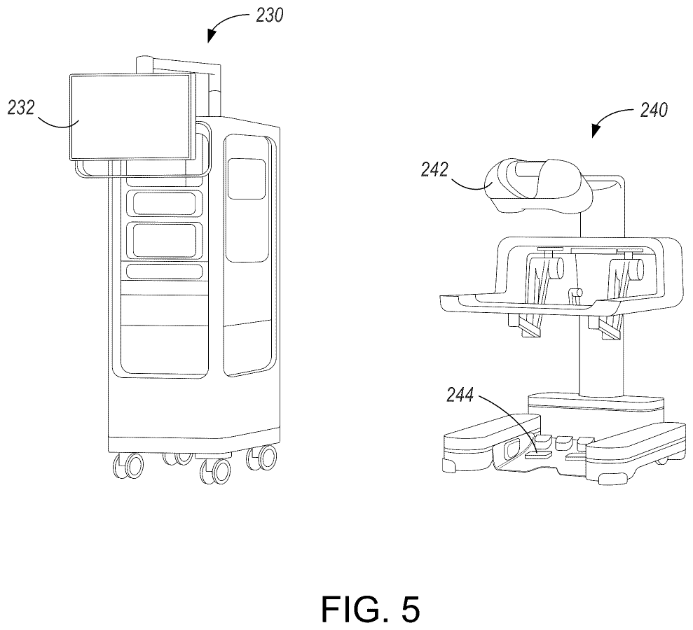

Referring now to FIGS. 6A-6C, different views of an exemplary robotic arm 210 are shown according to some embodiments. Particularly, in this exemplary embodiment, robotic arm 210 includes a plurality of links 302 (e.g., linkages), indicated as 302-1 through 302-4 in FIGS. 6A-6C, connected by one or more joints 304 (indicated as 304-1 through 304-6 in FIGS. 6A-6C). Each of the joints 304 includes one or more DoFs.

In FIG. 6A, the joints 304 include a first joint 304-1 (e.g., a base joint or an A0 joint) located at or near a base 306 of the robotic arm 210. In some embodiments, the base joint 304-1 comprises a prismatic joint that allows the robotic arm 210 to translate along the support rail 220 (e.g., along the y-direction indicated in FIG. 2). The joints 304 also include a second joint 304-2 which, in some embodiments, rotates with respect to the base joint 304-1. The joints 304 also include a third joint 304-3 that is connected to a first end of link 302-2 and which, in some embodiments, includes multiple DoFs and facilitates both tilt and rotation of the link 302-2 tilt with respect to the third joint 304-3. In addition, joints 304 include a fourth joint 304-4 that is connected to a second end of the link 302-2. In some embodiments, the joint 304-4 comprises an elbow joint that connects the link 302-2 to the link 302-3. In this exemplary embodiment, the joints 304 further include a pair of joints 304-5 (e.g., a wrist roll joint) and 304-6 (e.g., a wrist pitch joint), which is located on a distal portion of the robotic arm 210.

In this exemplary embodiment, a proximal end of the robotic arm 210 is connected to a base 306 and a distal end of the robotic arm 210 is connected to device manipulator or driver such as, for example, an advanced device manipulator (ADM) 308 (e.g., a tool driver, an instrument driver). The ADM 308 may be configured to control the positioning and manipulation of a medical instrument s (e.g., a tool, a scope, etc.). For example, in some embodiments, the links 302 may be detachably coupled to a medical tool 212 (e.g., to facilitate case of mounting and dismounting of the medical tool 212 from the robotic arm 210) carried by the ADM 308. The joints 304 provide the robotic arm 210 with a plurality of DoFs required to facilitate control of the medical tool 212 via the ADM 308.

The robotic arm 210 may also include a cannula sensor 310 for detecting the presence or proximity of a cannula to the robotic arm 210. In some embodiments, the robotic arm 210 is placed in a docked state (e.g., docked position) when the cannula sensor 310 detects the presence of a cannula (e.g., via one or more processors of the robotic medical system 200). Conversely, when no cannula is detected by the cannula sensor 310, the robotic arm 210 is placed in an undocked state (e.g., undocked position).

In some embodiments, and as shown particularly in FIG. 6A, the robotic arm 210 includes a control input or button 312 (e.g., an annular button, or other types of controls, etc.) that can be used to place the robotic arm 210 in an admittance mode (e.g., by depressing the button 312). The admittance mode is also referred to as an admittance scheme or admittance control. In the admittance mode, the robotic medical system 200 measures forces and/or torques (e.g., imparted on the robotic arm 210) and outputs corresponding velocities and/or positions. In some embodiments, the robotic arm 210 can be manually manipulated by a user (e.g., during a set-up procedure, or in between procedures, etc.) in the admittance mode. In some instances, by using the admittance mode, a user of robotic arm 210 need not overcome all of the inertia in the robotic medical system 200 to moves the robotic arm 210. For example, in the admittance mode, the robotic medical system 200 can measure the force and assist the operator in moving the robotic arm 210 by driving one or more motors associated with the robotic arm 210 when the operator imparts a force on the arm, thereby producing the desired velocities and/or positions of the robotic arm 210.

In some embodiments, the robotic arm 210 includes a second input or button 314 (e.g., a push button) that is distinct from the button 312 shown in FIG. 6A (referred to now as the first button 312), for placing the robotic arm 210 in an impedance mode (e.g., by a single press or continuous press and hold of the second button 314). The impedance mode is also referred to as impedance scheme or impedance control. In the impedance mode, the robotic medical system 200 measures displacements (e.g., changes in position and velocity) and outputs forces and/or torques to facilitate manual movement of the robotic arm 210. In some embodiments, the robotic arm 210 can be manually manipulated by a user (e.g., during a set-up procedure) in the impedance mode. In some embodiments, under the impedance mode, the operator's movement of one part of a robotic arm 210 may cause motion in one or more joints and/or links throughout the robotic arm 210.

In some embodiments, for admittance control, a force sensor or load cell measures the force that the operator is applying to the robotic arm 210 and move the robotic arm 210 in a way that feels light. Admittance control may feel lighter than impedance control because, under admittance control, one can hide the perceived inertia of the robotic arm 210 through the motorized actuation of the joints 304 thereof. Conversely, in some embodiments, the user is responsible for most if not all mass acceleration using impedance control.

In some circumstances, depending on the position of the robotic arm 210 relative to the user, it may be inconvenient to reach the button 312 and/or the button 314 to activate a manual manipulating mode (e.g., the admittance mode and/or the impedance mode). Accordingly, under these circumstances, it may be convenient for the operator to trigger the manual manipulation mode other than by buttons. Additionally, in some embodiments, the robotic arm 210 includes a single button (e.g., the button 312 or 314) that can be used to place the robotic arm 210 in the admittance mode and/or the impedance mode (e.g., by using different presses, such as a long press, a short press, press and hold etc.). In some embodiments, the robotic arm 210 can be placed in impedance mode by a user pushing on arm linkages (e.g., the links 302) and/or joints (e.g., the joints 304) and overcoming a force threshold. In some embodiments, the admittance mode and the impedance mode are common in that they both allow the user to grab the robotic arm 210 and command motion thereof by directly and physically interfacing with the robotic arm 210.

In some embodiments, the robotic arm 210 includes an input control for activating an arm follow mode. For example, in some embodiments, the robotic arm 210 includes a designated touch point located on a link 302 or a joint 304 of the robotic arm 210 (e.g., an outer shell of the link 302 or a button 316). User interaction (e.g., user touch) with the designated touch point activates the arm follow mode. In some embodiments, the robotic arm 210 includes multiple touch points in which user interaction with any (e.g., one or more) of the touch points activates the arm follow mode of the robotic arm 210

During a medical procedure, it may be desired to have the ADM 308 of the robotic arm 210 and/or a remote center of motion (RCM) of the medical tool 212 coupled thereto kept in a static pose (e.g., position and/or orientation). An RCM may refer to a point in space where a cannula or other access port through which a medical tool 212 is inserted is motion constrained. In some embodiments, the medical tool 212 includes an end effector that is inserted through an incision or natural orifice of a patient while maintaining the RCM. In some embodiments, the medical tool 212 includes an end effector that is in a retracted state during a setup process of the robotic medical system.

In some circumstances, the robotic medical system 200 may be configured to move one or more links 302 of the robotic arm 210 within a “null space” to avoid collisions with nearby objects (e.g., other robotic arms), while the ADM 308 of the robotic arm 210 and/or the RCM are maintained in their respective poses. The null space may be viewed as the set of joint states through which a robotic arm 210 is permitted to move which does not result in movement of the ADM 308 and/or RCM, thereby maintaining the position and/or the orientation of the medical tool 212 (e.g., within a patient). In some embodiments, a robotic arm 210 may have multiple positions and/or configurations available for each pose of the ADM 308.

For a robotic arm 210 to move the medical tool 212 to a desired pose in space, in certain embodiments, the robotic arm 210 is provisioned with at least six DoFs—three DoFs for translation (e.g., X, Y, and Z positions) and three DoFs for rotation (e.g., yaw, pitch, and roll). In some embodiments, cach joint 304 may provide the robotic arm 210 with a single DoF, and thus, the robotic arm 210 may have at least six joints to achieve freedom of motion to position the ADM 308 at any pose in space. To further maintain the ADM 308 or the RCM of the robotic arm 210 in a desired pose, the robotic arm 210 may further be provided with at least one additional “redundant joint.” Thus, in certain embodiments, the system may include a robotic arm 210 having at least seven joints 304, providing the robotic arm 210 with at least seven DoFs. In some embodiments, the robotic arm 210 may include a subset of joints 304 cach having more than one DoF thereby achieving the additional DoFs for null space motion. Depending on the embodiments, the robotic arm 210 may have a greater or fewer number of DoFs.

Furthermore, in some embodiments, the support rail 220 may provide several additional DoFs, including lift, lateral translation, tilt, etc. Thus, depending on the embodiments, a robotic medical system may have many more robotically controlled DoFs beyond just those in the robotic arms 210 to provide for null space movement and/or collision avoidance. In a respective embodiment of these embodiments, the end effectors of one or more robotic arms (and any tools or instruments coupled thereto) and a remote center along the axis of the tool can advantageously maintain in pose and/or position within a patient. Additionally, a robotic arm 210 having at least one redundant DoF has at least one more DoF than the minimum number of DoFs for performing a given task. For example, a robotic arm 210 can have at least seven DoFs, where one of the joints 304 of the robotic arm 210 can be considered a redundant joint, in accordance with some embodiments. The one or more redundant joints can allow the robotic arm 210 to move in a null space to both maintain the pose of the ADM 308 and a position of an RCM and avoid collision(s) with other robotic arms or objects.

Referring now to FIGS. 7A and 7B, an exemplary robotic arm 350 is shown in accordance with some embodiments. In this exemplary embodiment, robotic arm 350 includes a tool drive 352 and a cannula 362 loaded with a robotic surgical tool. Additionally, robotic arm 350 includes links 351 (e.g., link 351-1, link 351-2, link 351-3, link 351-4, and/or link 351-5) and actuated joint modules (e.g., a joint 353, see also joints J1, J2, J3, J4, J5, J6, J7, and J8) for actuating the plurality of links 351 relative to one another. The joint modules may include various types, such as a pitch joint or a roll joint, which may substantially constrain the movement of the adjacent links around certain axes relative to others. In this exemplary embodiment, a tool drive 352 is attached to the distal end of the robotic arm 350, the tool drive 352 including a carriage 354 and a stage 356.

As shown particularly in FIG. 7A, in some embodiments, tool drive 352 includes the cannula 362 coupled to its end to receive and guide a surgical instrument 360 (e.g., endoscopes, staplers, scalpel, scissors, clamp, retractor, etc.). The surgical instrument (or “tool”) 360 may include an end effector 364 at the distal end of the tool 360. The plurality of the joint modules of the robotic arm 350 may be actuated to position and orient the tool drive 352, which actuates the end effector 364 for robotic surgeries. The end effector 364 is located at a tool shaft end or “tool tip” 365 of the tool 360. In other embodiments, the tool shaft end 365 is a tip of a needle or other object. In addition, in this exemplary embodiment, tool drive 352 includes a cannula release lever 358 for releasing the cannula 362 from the tool drive 352.

In some embodiments, the robotic arm 350 includes input devices (e.g., buttons, touchpoints, etc.) For example, as shown particularly in FIG. 7B, robotic arm 350 includes a clearance adjustment touchpoint 366, an instrument clutch 368, a port clutch 370, a forearm pivot touchpoint 372, and a forearm multipoint touchpoint 374.

Referring to FIGS. 7A, 7B, and 8, link 351-1 includes a first end that is coupled to the joint J1. In some embodiments, the robotic arm 350 includes a J0 joint that actuates a second end of the link 351-1. In certain embodiments, joint J0 is a table pivot joint and resides under the surgical table top 402 (shown in FIGS. 3A and 3B). In this exemplary embodiment, joint J0 is nominally held in place during surgery while joints J1 to J5 form a setup or Cartesian arm and are nominally held in place during surgery. Additionally, joints J6 and J7 (shown in FIG. 8) form a spherical arm that may actively move during surgery or teleoperation. Joint J8 translates the tool 360, such as the end effector 364, as part of a tool driver, where it may be understood that joint J8 may actively move during surgery. Further, joints J6, J7, and J8 actively position the tool tip 365 and end effector 364 during surgery while maintaining an entry point into the patient at a fixed or stable location (e.g., an RCM) to avoid stress on the body wall of the patient.

During setup of robotic arm 350, any of the joints J0-J8 are permitted to move. However, during surgery, the joints J6, J7, and J8 may move subject to hardware or safety limitations on position, velocity, acceleration, and/or torque. Additionally, the surgical tool 360 may include none, one, or more (e.g., three) joints, such as a joint for tool rotation plus any number of additional joints (e.g., wrists, rotation about a longitudinal axis, or other type of motion). In this manner, any number of DoFs may be provided, such as the three degrees from the joints J6, J7, and J8 and none, one, or more degrees from the surgical tool 360.

FIG. 8 illustrates part of the robotic arm 350 and the surgical tool 360 providing six DoFs. The six DoFs correspond to movement of six active joints during teleoperation. In some embodiments, the active joints include three joints on the surgical tool 360—rotation at joint J9, pitch at wrist joint J10, and yaw at wrist joint J11. The active joints may include three joints on the robotic arm 350—spherical roll joint J6, spherical pitch joint J7, and tool translation joint J8. In some embodiments, other joints providing the six DoFs may be used.

In some embodiments. a user may command movement of fewer than six DoFs. For example, in some embodiments, five, four, or even fewer active joints may be provided. In an example of five DoFs, the active joints include three joints on the robotic arm 350—spherical roll joint J6, spherical pitch A joint J7, and tool translation joint J8—and two active joints on the surgical tool—rotation at joint J9 and articulation as another joint. In some embodiments, active joint arrangements may be used, such as providing two or fewer DOF on the robotic arm during teleoperation.

### C. Exemplary Surgical Settings

Referring now to FIG. 9, a perspective view of an embodiment of a robotic medical system 450 including a pair of support rails 220-1 and 220-2, and four of the robotic arms 210-1, 210-2, 210-3, and 210-4 is shown. In this exemplary embodiment, each of the robotic arms 210-1, 210-2, 210-3, and 210-4 is coupled to a respective surgical tool 452 (e.g., 452-1 through 452-4, which may correspond to medical tool 212) via a respective ADM 308 (e.g., tool driver), such as ADMs 308-1 through 308-4. One or more of the surgical tools 452-1, 452-2, 452-3, and 452-4 may be inserted into a patient via one or more corresponding ports 458 (e.g., 458-1, 458-2, 458-3, 458-4, and 458-4) located on the patient. As used herein, a port (e.g., a port location, a port of entry, an entry point, a port region, a port area, or a port position, etc.) refers to a position on a patient's body through which a medical tool/instrument (e.g., held by a robotic arm) can be inserted and constrained in motion. In some embodiments, the port corresponds to an incision point or region that is made through the skin of the patient to facilitate a medical operation or procedure. In some embodiments, the port corresponds to a natural orifice, such as a mouth of the patient (e.g., for a bronchoscopy procedure). In some embodiments, the port corresponds to a medical device with an opening, placed at the incision point or the natural orifice to allow access to a surgical space through the opening. The view of the patient has been excluded from FIG. 9 in order to enhance the visibility of the robotic arms 210-1, 210-2, 210-3, and 210-4 and the surgical tools 452-1, 452-2, 452-3, and 452-4.

In this exemplary embodiment, the robotic arm 210-2 is coupled to a camera or scope 456 via a medical instrument 452-2 (e.g., an endoscope). In some embodiments, the camera 456 is a part of the medical instrument 452-2. In some embodiments, the camera 456 can be a standalone device (e.g., not part of a medical instrument) that is coupled to a robotic arm. In some embodiments, the camera 456 defines an axis 454 (e.g., an optical axis), which identifies an orientation of the camera 456. The camera 456 (or a scope) provides an image of a surgical site to facilitate control of surgical tools to perform a robotic medical procedure. For example, a robotically controllable endoscope of the robotic medical system 450 may include a camera positioned at a distal tip thereof. The user can view an image from the camera of the endoscope in a viewer in order to facilitate control of the endoscope and/or other components of the robotic medical system 450. As another example, the robotic medical system 450 may include one or more cameras or other sensors laparoscopically or endoscopically inserted into a patient. A user of robotic medical system 450 may view images from the inserted cameras in order to facilitate control of one or more additional robotically controlled medical instruments, such as one or more additional laparoscopically inserted medical instruments.

In some embodiments, the robotic medical systems disclosed herein (e.g., robotic medical systems 200, 250, 400 450) includes a coordinate system (e.g., a robot coordinate system, a coordinate frame, a system frame, etc. that may be a Cartesian or non-Cartesian coordinate system), and respective positions of the patient platform 202, the table top 402, the robotic arms 210, the robotic arms 350, the support rails 220, and/or medical tools 212 are represented as coordinates (e.g., x-, y-, and z-coordinates) on the coordinate system. For example, robotic medical systems disclosed herein (e.g., one or more processors 380 of the robotic medical system 200 or the robotic medical system 400) may be configured to identify positions and orientations of the patient platform 202, the table top 402, the robotic arms 210, the support rails 220, and/or medical tools 212 based on coordinates in the coordinate system.

### D. Contact Forces Applied by Robotic Arms of a Surgical Robot

The robotic arms of surgical robots (e.g., robotic medical systems) may, in the course of facilitating a teleoperation, contact or apply contact forces to different objects encountered by the respective robotic arm. For example, a robotic arm may apply a contact force to a body wall of a patient when a medical tool (e.g., medical tool 212) of the robotic arm is inserted into the body of the patient, where the contact force may result from relative motion between the robotic arm and the body of the patient. As another example, a first robotic arm of a surgical robot may apply a contact force to a second robotic arm of the surgical robot in response to a collision between the first robotic arm and the second robotic arm.

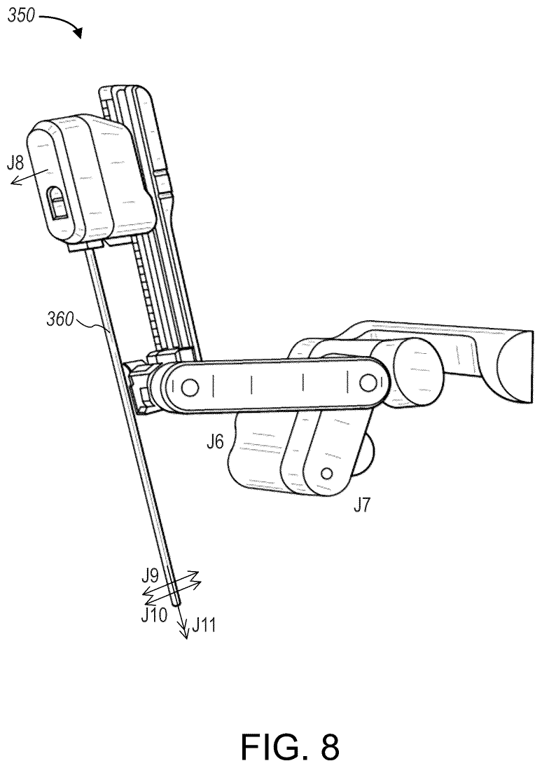

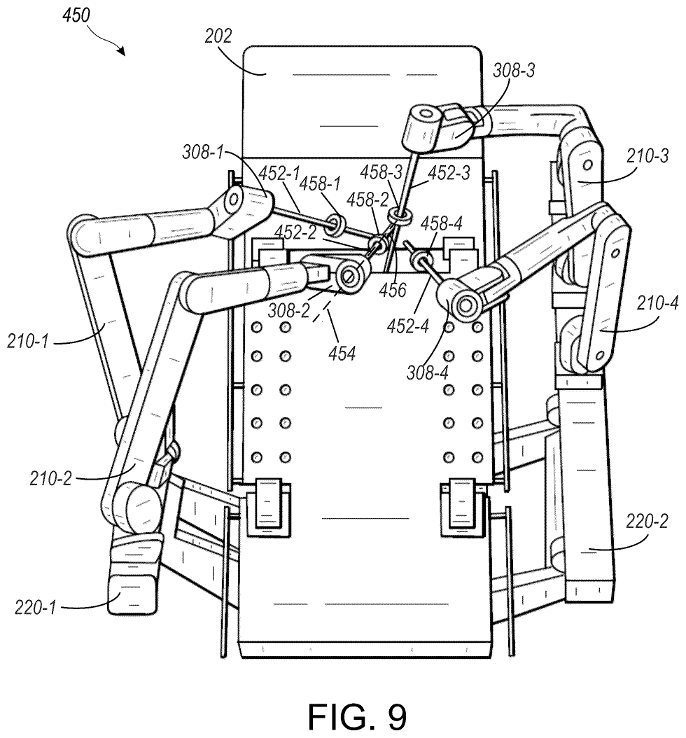

Referring now to FIGS. 10 and 11, a robotic arm 510 of a surgical robot 500, according to some embodiments is partially shown, being inserted into a body 522 of a patient 520 via a cannula 530 inserted into a body wall 524 of the patient 520. Particularly, the robotic arm 510 includes an ADM 512 at a distal end thereof and a medical tool or shaft 514. The medical tool 514 extends from the ADM 512, through the cannula 530 located along the body wall 524 of patient 520, to a distal end of the medical tool 514 at which an end effector 516 of the medical tool 514 is located.

In this example the RCM 532 (shown in FIG. 11) of robotic arm 510 is defined by and located along the cannula 530 to minimize relative motion between the medical tool 514 of robotic arm 510 and the body 522 of patient 520. In this configuration with the RCM 532 located at the cannula 530, the medical tool 514 of robotic arm 510 is permitted to move bi-directionally along a linear or rectilinear insertion axis 515 (shown in FIG. 10) of the robotic arm 510 in response to the actuation of one or more of the actuators or joints of the robotic arm 510. Additionally, the medical tool 514 is permitted, while maintaining the RCM 532 located at the cannula 530, to both pitch (indicated by arrow 517 in FIG. 10) and yaw (indicated by arrow 519 in FIG. 10) relative to the body 522 of patient 520. In other embodiments the medical tool 514 may be additionally constrained such that the medical tool is prevented from, for example pitching 517 or yawing 519 relative to the body 522 of patient 520. Additionally, in other embodiments, additional DoFs may be permitted between the medical tool 514 and body 522 of patient 520. For instance, the medical tool 514 may be permitted to rotate about the insertion axis 515 in some embodiments.

Robotic arm 510 applies a contact force (e.g., indicated by arrow 521 in FIG. 11) to the body 522 of patient 520 (e.g., body tissue 526 shown schematically in FIG. 11) in response to the relative motion (indicated by arrow 523 in FIG. 11) between the end effector 516 of medical tool 514 and the body 522 of patient 520 that occurs as the robotic arm 510 performs a clinical task. Additionally, robotic arm 510 may apply contact forces via the cannula 530 to the body 522 of patient 520 as the robotic arm 510 performs a clinical task. The external loads applied to the robotic arm 510 (e.g., reactive loads arising from, for example, contact force 521) resulting from contact between the robotic arm 510 and the body 522 of patient 520 (e.g., between the end effector 516 and body tissue 526 of the body 522) as well as the loads applied to the robotic arm 510 through the cannula 530 from the body wall 524 comprise expected clinical loads or forces encountered by the robotic arm 510 as part of performing a clinical task.

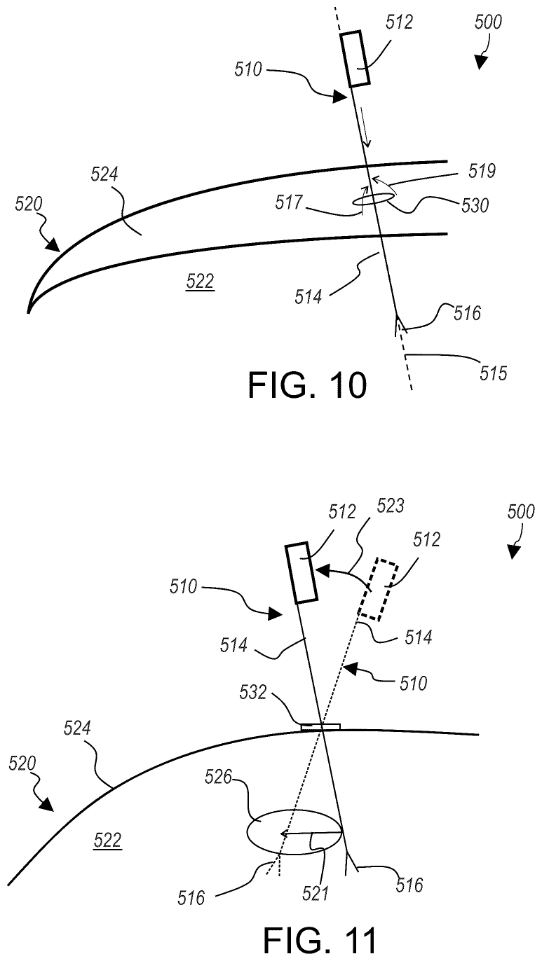

In addition to the clinical loads described above, robotic arm 510 may also encounter other external loads or forces during a teleoperation such as unexpected external loads resulting from, for example, a collision between the robotic arm 510 and an external object. For example, referring to FIG. 12, the robotic arm 510 of FIG. 10 is shown contacting an external object 540 (shown schematically in FIG. 12) that is external of and separate from the body 522 of patient 520 whereby the robotic arm 510 applies a contact force (indicated by arrow 542 in FIG. 12) to the external object 540. For example, during the performance of a teleoperation, the robotic arm 510 may inadvertently collide with another or second robotic arm 510 (or other components of surgical robot 500) of surgical robot 500 whereby the contact force 542 is applied to the second robotic arm 510. As another example, during the performance of a teleoperation, the robotic arm 510 may inadvertently collide with a clinician.

As outlined above, robotic arm 510 may apply both expected contact forces (e.g., to the body tissue 526 as part of performing a clinical task) and unexpected contact forces (e.g., as the result of a collision) to external objects during the performance of a teleoperation. Robotic arm 510 may apply said contact forces when both actively moving (e.g., moving the end effector 516 through the body 522 of patient 520) and when in a stationary state where it is intended for the robotic arm 510 to remain stationary with respect to a proximal end of the robotic arm 510 coupled to a base or other support structure (e.g., as a result of an external object colliding with the stationary robotic arm 510).

Excessive contact force applied by the robotic arm 510 to external objects (e.g., body tissue 526 of body 522, other components of surgical robot 500) during a teleoperation facilitated by the surgical robot 500 could pose a safety risk to the patient 520 and the clinicians operating surgical robot 500, as well as a risk of damaging equipment of surgical robot 500.

In addition to the risks posed by excessive contact force applied by the robotic arm 510, a safety risk may be posed to the patient 520 and/or clinicians operating the surgical robot 500 in the event of the robotic arm 510, when in a stationary state, is inadvertently moved in response to a collision between the stationary robotic arm 510 and an external object (e.g., another robotic arm 510 of surgical robot 500, a user of surgical robot 500). For example, the end effector 516 coupled to the robotic arm 510 may perform a clinical task by precisely gripping and statically positioning a specific body tissue 526 of the body 522 of patient 520. A safety risk may be posed to the patient 520 in this example if the stationary robotic arm 510 were to be inadvertently moved (resulting in undesired movement of the end effector 516 gripping the body tissue 526 of patient 520 and/or the end effector 516 inadvertently piercing or cutting body tissue 526) in response to a collision between the robotic arm 510 and an external object 540.

### E. Systems for Applying Torque Limits to Robotic Arms of Surgical Robots

To address the safety risks posed by excessive contact force applied by robotic arms of surgical robots (e.g., robotic medical systems such as, for example, robotic medical systems 200, 250, 400, and/or 450) to external objects and/or inadvertent movement of a stationary robotic arm resulting from a collision between the robotic arm and an external object, embodiments of surgical robots are disclosed herein which apply torque limits to the robotic arms (e.g., robotic arms 210, 350) thereof to limit the contact forces that may be applied by the robotic arms and to prevent inadvertent movement of a stationary robotic arm in response to a collision between the stationary robotic arm and an external object.

In some embodiments, a surgical robot comprising a plurality of robotic arms (e.g., a plurality of robotic arms 210 and/or 350) apply a first or dynamic torque limit to one or more joints of a respective robotic arm when the robotic arm is actively moving, and apply a second or static torque limit which is independent from the dynamic torque limit to one or more joints of a respective robotic arm when the robotic arm is in a stationary state. Particularly, the dynamic torque limit is intended to allocate relatively more torque to the stationary robotic arm than the actively moving robotic arm for counteracting unexpected external loads applied to the stationary robotic arm. Thus, while the static torque limit may exceed the dynamic torque limit in many instances, there may be some instances in which the static torque limit is equal to or less than the dynamic torque limit when the expected external loads (e.g., gravitational external loads contingent upon the respective poses of the stationary and actively moving robotic arms) applied to the actively moving robotic arm exceed the expected external loads applied to the stationary robotic arm.

In this manner, a first robotic arm of the surgical robot in the stationary state may remain stationary (e.g., an end effector coupled to the stationary robotic arm may remain stationary) in spite of being collided with by a second robotic arm of the surgical robot that is actively moving. As used herein, the term “actively moving” refers to a robotic arm that is currently or actively moving with respect to a support structure that physically supports the proximal end of the robotic arm such as, for example a patient platform (e.g., patient platform 202), a table, a bed. A robotic arm is actively moving when any component of the robotic arm is actively moving with respect to the support structure. Additionally, a robotic arm is actively moving when an end effector coupled to the robotic arm is actively moving with respect to the support structure.

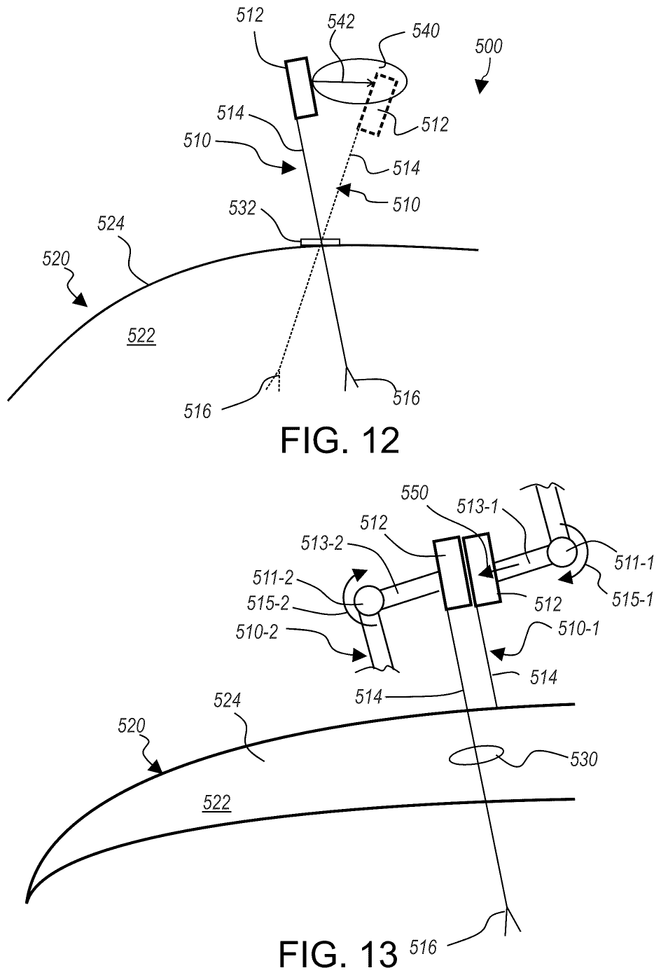

As an example, and referring now to FIG. 13, surgical robot 500 is shown with a first robotic arm 510-1 thereof that is actively moving and a second robotic arm 510-2 thereof that is in a stationary state. In this example, cach robotic arm 510-1 and 510-2 includes an actuator or joint 511 and a link 513 coupled between the joint 511 and the ADM 512, where the link 513 is driven by the joint 511. Particularly, the joint 511-1 of robotic arm 510-1 applies a torque (indicated by arrow 515-1 in FIG. 13) to the link 513-1 and ADM 512 thereof which, in this example, results in a collision (indicated by arrow 550 in FIG. 13) between the ADM 512 of robotic arm 510-1 and the ADM 512 of robotic arm 510-2. The collision between robotic arms 510-1 and 510-2 results in the application of a contact force (indicated by arrow 550 in FIG. 13) by the robotic arm 510-1 to the robotic arm 510-2.

In addition, in this example, the joint 511-2 of robotic arm 510-2 automatically applies a counteracting or resistive torque (indicated by arrow 515-2 in FIG. 13) to the link 513-2 to resist the contact force 550 applied to the robotic arm 510-2 by the robotic arm 510-2. In some embodiments, a processor of surgical robot 500 (e.g., processor 380) automatically applies the resistive torque 515-2 to the link 513-2 of robotic arm 510-2 in response to detecting the contact force 550 so as to maintain robotic arm 510-2 in the previously established stationary state. In other words, the processor of surgical robot 500 is configured, in this example, to automatically apply resistive torques to the joints 511 of a respective robotic arm 510 when the robotic arm 510 is in a stationary state so as to prevent the respective robotic arm 510 from moving in response to the application of the contact force thereto.

In some embodiments, the processor of surgical robot 500 applies a dynamic torque limit to one or more of the joints 511 of a given robotic arm 510 when the respective robotic arm 510 is actively moving (e.g., relative to a support structure physically supporting proximal ends of robotic arm 510). Additionally, in some embodiments, the processor applies a static torque limit to one or more of the joints 511 of a given robotic arm 510 when the robotic arm 510 is in a stationary state. For example, the processor may continuously apply the dynamic torque limit to each of the robotic arms 510 of surgical robot 500 that are actively moving, while simultaneously continuously applying the static torque limit to each of the robotic arms 510 of surgical robot 500 that are in a stationary state. The processor may automatically switch from applying the dynamic torque limit to applying the static torque limit to a given robotic arm 510 in response to the robotic arm 510 entering a stationary state. Conversely, the processor may automatically switch from applying the static torque limit to applying the dynamic torque limit to a given robotic arm 510 in response to the robotic arm 510 beginning to actively move.

By applying a different torque limit to the robotic arms 510 in a stationary state than the robotic arms 510 that are actively moving, the robotic arms 510 in the stationary state may successfully resist moving in response to the application of a contact force to the respective robotic arm 510 from an external object. In other words, the heightened torque limit applied to the robotic arm 510 by the processor permits the robotic arm 510 to remain in the stationary state even when a contact force is applied to the stationary robotic arm 510 from an external object.

With reference to the example presented in FIG. 13, the dynamic torque limit is applied by the processor to the joint 511-1 of robotic arm 510-1 given that robotic arm 510-1 is actively moving. Additionally, the static torque limit is applied by the processor to the joint 511-2 of robotic arm 510-2 given that robotic arm 510-2 is in a stationary state. In this manner, the processor may apply as much resistive torque 515-2 to the link 513-2 of robotic arm 510-2 as needed to maintain the robotic arm 510-2 (along with the end effector 516 coupled thereto) in the stationary state such that the robotic arm 510-2 does not move in response to the application of contact force 550 by robotic arm 510-1.

In certain embodiments, the dynamic torque limit applied by the processor of surgical robot 500 to the robotic arms 510 thereof that are actively moving is based on an expected torque estimated by the processor as required by one or more of the joints 511 of the respective robotic arm 510 to satisfy a control input applied to the robotic arm 510 (e.g., by a user of surgical robot 500 via a physician console 240 of surgical robot 500 as part of the performance of a clinical task using the robotic arm 510). In other words, in order to satisfy a control input applied by the robotic arm 510, one or more of the joints 511 of the robotic arm 510 must apply one or more respective torques in order for the robotic arm 510 to move in accordance with the given control input. For instance, the one or more respective torques of the one or more joints 511 are necessary to overcome the force of gravity applied to the links 513 of the robotic arm 510, frictional torque applied to one or more of the joints 511 of the robotic arm 510 in response to movement thereof, and other drags applied to the robotic arm 510 such as tissue contact forces and the like applied to end effector 516, for example.

The magnitude of such torque (e.g., torques resulting from gravitational forces, frictional drags) vary in magnitude depending on the given pose of the robotic arm 510 and the type of control input applied to the robotic arm 510. For example, the initial pose of the robotic arm 510 prior to the application of the control input will influence the gravitational forces applied to the links 513 of the robotic arm 510. Additionally, motion of the links 513 of robotic arm 510 following execution of the control input may also influence the frictional drag applied to the robotic arm 510.

In certain embodiments, the expected torque estimated by the processor is based on a predefined dynamic model of the respective robotic arm 510. For example, a dynamic model of a given robotic arm 510 may be stored in memory (e.g., memory 382) of the surgical robot 500 whereby a process (e.g., processor 380) of surgical robot 500 may access and utilize the dynamic model of the robotic arm 510 to estimate the expected torques required of one or more of the joints 511 of the robotic arm 510 to accomplish or satisfy a given control input applied to the robotic arm 510.

Not intending to be bound by any particular theory, Equation (1) below represents an exemplary expected torque (τ_{E}) produced by a joint of a robotic arm (e.g., a joint 511 of robotic arm 510) and which corresponds to the sum of the frictional loads (frictional torque or τ_{F}), gravitational loads (gravitational torque or τ_{G}), and dynamic loads (dynamic torque or τ_{D}) imposed on the given joint during actuation.

$${τ}_{E} = {τ}_{G} + {τ}_{F} + {τ}_{D} \qquad ( 1 )$$

Equation (1) above may represent a feature or component of a dynamic model of the joint of the given robotic arm (e.g., a joint 511 of robotic arm 510). The expected torque (τ_{E}) of Equation (1) does not account for unknown, indeterminate external loads such as loads applied to the robotic arm from external objects (e.g., loads resulting from contact between an end effector coupled to the robotic arm and tissues of a given patient, loads resulting from a collision between the robotic arm and an external object).

In some embodiments, along with estimating an expected torque for one or more joints 511 of robotic arm 510, the processor of surgical robot 500 also estimates an error of the expected torque (e.g., an expected torque error) which may be based, at least in part, on an estimated accuracy of the dynamic model. For example, and not intending to be bound by any particular theory, Equation (2) below presents an exemplary expected torque error (E_{τ}) corresponding to the sum of the products of (τ_{G}) multiplied by a gravity accuracy constant (A_{G}), (τ_{F}) multiplied by a friction accuracy constant (A_{F}), and (τ_{D}) multiplied by a dynamic accuracy constant (A_{D})

$${E}_{τ} = {A}_{G} \times {τ}_{G} + {A}_{F} \times {τ}_{F} + {A}_{D} \times {τ}_{D} \qquad ( 2 )$$

In certain embodiments, along with estimating both the expected torque (τ_{E}) and error of the expected torque (E_{τ}), the processor of surgical robot 500 additionally estimates the contribution to the expected torque (τ_{E}) provided by expected external or clinical loads applied to the robotic arm 510 as it moves in response to the application of a control input thereto. As described above, expected clinical loads include, for example, loads resulting from normal contact between the end effector 516 coupled to robotic arm 510 and the body tissue 526 of the patient 520). Additionally, expected clinical loads may be influenced by or contingent on temporal variables such as the pose of the robotic arm 510.

By estimating the contribution provided by expected clinical loads, the processor of surgical robot 500 may, in some embodiments, additionally estimate an expected torque applied to one or more joints 511 of robotic arm 510 resulting from contact between the robotic arm 510 (including the end effector 516 coupled therewith) and external objects.

In some embodiments, the processor of surgical robot 500 estimates an expected torque, such as a bounded expected torque (E_{τB}), that defines both an upper bound (e.g. E_{τU}) and a lower bound (e.g. E_{τL}) where the upper bound (E_{τU}) is greater than lower bound (E_{τL}). In some instances, the expected torque may comprise a nominal or initial expected torque estimated by the processor while the bounded expected torque may comprise a final expected torque estimated by the processor that is based on the initial expected torque.

As an example, and not intending to be bound by any particular theory, Equation (3) below presents an exemplary bounded expected torque (E_{τB}) where (τ_{tissueMax}) represents the estimated maximum expected tissue load applied to a given joint of a robotic arm (e.g., a maximum τₜᵢₛₛᵤₑ):

$${E}_{τ B} = ∈ [ {E}_{τ} - {τ}_{tissueMax} , {E}_{τ} + {τ}_{tissueMax} ] \qquad ( 3 )$$

The processor of surgical robot 500 may continuously estimate the expected torque (e.g., E_{τB}) for one or more joints 511 of the robotic arm 510 in real-time, with the expected torque varying dynamically in response to, at least in part, changes to the control input applied to the robotic arm 510. Thus, in at least some embodiments, the expected torque estimated by the processor for a given joint 511 may vary continuously over time in response to changing conditions.

In addition to estimating the expected torque or bounded expected torque as described above, the processor also applies, based on the estimated expected torque, a dynamic torque limit to one or more joints 511 of the robotic arm 510 that is based on the corresponding expected torques (e.g., E_{τB}) of the one or more joints 511 estimated by the processor. For example, based on Equation (3) above, the processor may set a dynamic torque limit for a joint 511 that is equal to or greater than the current upper bound expected torque (E_{τU}) for the joint 511 as estimated by the processor, where the upper bound expected torque (and hence the dynamic torque limit) may change continuously over time in response to changing conditions (e.g., changes to the control input, changes to the pose of the robotic arm 510). In some embodiments, the dynamic torque limit set by the processor comprises a bounded torque limit that is bounded between an upper bound torque limit and a lower bound torque limit whereby a respective joint 511 is limited to torques falling between the lower bound torque limit and the upper bound torque limit.

In some embodiments, the dynamic torque limit applied to the joint 511 by the processor is equal to the upper bound expected torque (E_{τU}) for the joint 511. The processor of surgical robot 500 may determine a current to be supplied to the joint 511 (e.g., to an actuator or motor of the joint 511) commensurate with the upper bound expected torque (E_{τU}) using a correction factor saved in memory of the surgical robot 500. In other words, the torque limits (be they dynamic or static) applied by the processor of surgical robot 500 to one or more joints 511 of robotic arm 510 may first be transcribed by the processor into electrical currents to be supplied to the one or more joints 511 based on one or more corresponding correction factors or other information stored in the memory of the surgical robot 500.

In certain embodiments, the dynamic torque limit applied to the joint 511 by the processor is equal to either a product of the upper bound expected torque (E_{τU}) for the joint 511 and a correction factor which may, in some embodiments, account for the expected torque error (e.g., E_{τ}) inherent in the dynamic model of the robotic arm 510. In other words, the correction factor (which may be dynamic or predefined) may be correlated with the expected torque error such that an increase in the expected torque error results in a corresponding increase in the correction factor. However, in other embodiments, the correction factor may not be based or otherwise associated on the expected torque error.

In certain embodiments, the correction factor is tunable by a user of surgical robot 500 depending on the given needs of the user. For example, the user may increase the correction factor (e.g., continuously or between two or more predefined settings) to correspondingly increase the dynamic torque limit applied to one or more joints 511 of a given robotic arm 510. For example, in some instances, a user as part of performing a clinical task, may wish to drive a given robotic arm 510 through an external obstruction. In such a scenario, the user may increase (e.g., via physician console 240 or another user interface of surgical robot 500) the correction factor for the respective robotic arm 510 to correspondingly increase the dynamic torque limit applied by the processor to the respective robotic arm 510 thereby permitting, in this example, the user to drive the robotic arm 510 through the given external obstruction.

However, in at least some embodiments, a maximum dynamic torque limit that may be applied by the processor to a moving robotic arm 510 corresponds to a predefined maximum permissible force that the robotic arm 510 is permitted to apply to an object external the robotic arm. In other words, while a user of surgical robot 500 may have some degree of control over the dynamic torque limit (e.g., via adjusting the correction factor), in at least some instances the processor may not permit the application of a torque by one or more joints 511 of an actively moving robotic arm 510 that would result in the application of a force to an external object that exceeds the predefined maximum permissible force. The maximum permissible force may correspond to relevant safety standards or may be determined by a provider of the surgical robot 500. In some instances the processor may correlate a torque applied by one or more joints 511 of a robotic arm 510 with a contact force that may be applied by the robotic arm 510 as a result of the application of the torques by the one or more joints 511. For instance, the processor may correlate torques produced by joints 511 with a contact force applied by robotic arm 510 using the dynamic model of the robotic arm 510 stored in the memory of surgical robot 500.

### 3. Implementing Systems and Terminology

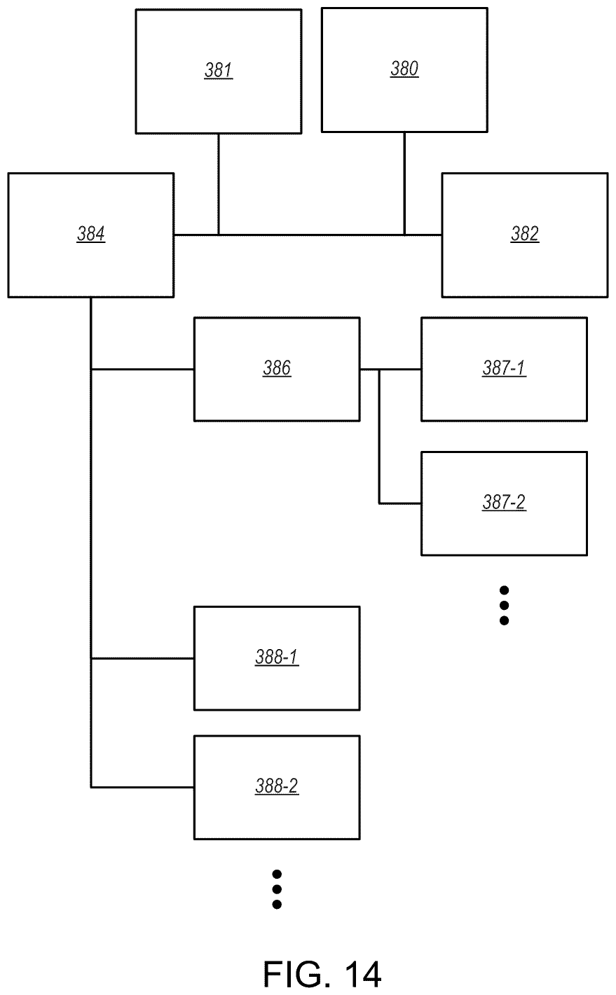

Referring to FIG. 14, a schematic diagram illustrating electronic components of a robotic medical system (e.g., a surgical robot) is shown in accordance with some embodiments.

The robotic medical system (e.g., surgical robot) includes one or more processors 380, which are in communication with a computer-readable storage medium 382 (e.g., computer memory devices, such as random-access memory, read-only memory, static random-access memory, and non-volatile memory, and other storage devices, such as a hard drive, an optical disk, a magnetic tape recording, or any combination thereof) storing instructions for performing any methods described herein (e.g., operations described with respect to FIGS. 1, 2, 3A, 3B, 4, 5, 6A, 6B, 6C, 7A, 7B, 8-13). The one or more processors 380 are also in communication with an input/output controller 384 (via a system bus or any suitable electrical circuit). The input/output controller 384 receives sensor data from one or more sensors 388-1, 388-2, etc., and relays the sensor data to the one or more processors 380. The input/output controller 384 also receives instructions and/or data from the one or more processors 380 and relays the instructions and/or data to one or more actuators, such as first motors 387-1 and 387-2, etc. In some embodiments, the input/output controller 384 is coupled to one or more actuator controllers 386 and provides instructions and/or data to at least a subset of the one or more actuator controllers 386, which, in turn, provide control signals to selected actuators. In some embodiments, the one or more actuator controllers 386 are integrated with the input/output controller 384 and the input/output controller 384 provides control signals directly to the one or more actuators 387-1 and 387-2 (without a separate actuator controller). Although FIG. 14 shows that there is one actuator controller 386 (e.g., one actuator controller for the entire medical robotic system, in some embodiments, additional actuator controllers may be used (e.g., one actuator controller for each actuator, etc.). In some embodiments, the one or more processors 380 are in communication with one or more displays 381 for displaying information as described herein.

The terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The functions for determining whether a tool is within or outside a surgical field of view provided by a camera or scope and rendering one or more indicators representing positions or directions of one or more medical tools described herein may be stored as one or more instructions on a processor-readable or computer-readable medium. The term “computer-readable medium” refers to any available medium that can be accessed by a computer or processor. By way of example, and not limitation, such a medium may comprise random access memory (RAM), read-only memory (ROM), electrically erasable programmable read-only memory (EEPROM), flash memory, compact disc read-only memory (CD-ROM) or other optical disk storage, magnetic disk storage or other magnetic storage devices, or any other medium that can be used to store desired program code in the form of instructions or data structures and that can be accessed by a computer. A computer-readable medium may be tangible and non-transitory. As used herein, the term “code” may refer to software, instructions, code or data that is/are executable by a computing device or processor.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or implementations.

As used herein, the term “and/or” encompasses any combination of listed elements. For example, “A, B, and/or C” includes the following sets of elements: A only, B only, C only, A and B without C, A and C without B, B and C without A, and a combination of all three elements, A, B, and C.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the scope of the disclosure. For example, it will be appreciated that one of ordinary skill in the art will be able to employ a number corresponding alternative and equivalent structural details, such as equivalent ways of fastening, mounting, coupling, or engaging tool components, equivalent mechanisms for producing particular actuation motions, and equivalent mechanisms for delivering electrical energy. Thus, the present disclosure is not intended to be limited to the embodiments shown herein but is to be accorded the widest scope consistent with the principles and novel features disclosed herein.

What is claimed is:

1. A surgical robot, comprising:
an end effector driven by a plurality of joints located along a robotic arm of the surgical robot; and
a processor communicatively coupled to the robotic arm, the processor configured to:
apply a first torque limit to at least one of the plurality of joints when the robotic arm is actively moving; and
apply a second torque limit to at least one of the plurality of joints when the robotic arm is in a stationary state, wherein the second torque limit is different from the first torque limit.

2. The surgical robot of claim 1, wherein the processor is configured to determine the first torque limit using a dynamic model of the robotic arm.

3. The surgical robot of claim 1, wherein the processor is configured to estimate an expected torque producible by the plurality of joints to satisfy a control input applied to the robotic arm, wherein the expected torque is based on a predefined dynamic model of the robotic arm.

4. The surgical robot of claim 3, wherein the expected torque comprises a sum of a plurality of torques required to overcome gravitational loads applied to the robotic arm, frictional loads applied to one or more of the plurality of joints, and dynamic loads resulting from movement of one or more joints of the robotic arm.

5. The surgical robot of claim 3, wherein the expected torque includes an estimate of an error of the predefined dynamic model upon which the expected torque is based.

6. The surgical robot of claim 5, wherein the expected torque comprises an estimated expected tissue load applied to the robotic arm during performance of a clinical task.

7. The surgical robot of claim 3, wherein the expected torque comprises an initial expected torque and the processor is configured to determine a final expected torque range that is bounded between an upper bound and a lower bound that is less than the upper bound.

8. The surgical robot of claim 1, wherein the first torque limit corresponds to a maximum permissible force that the robotic arm is able to apply to an object external to the robotic arm.

9. The surgical robot of claim 1, wherein the processor is configured to determine when the robotic arm is actively moving, and to determine when the robotic arm is in the stationary state.

10. The surgical robot of claim 1, wherein the second torque limit is greater than the first torque limit.

11. A surgical robot, comprising:
an end effector driven by a plurality of joints of a robotic arm of the surgical robot; and
a processor communicatively coupled to the robotic arm, the processor configured to:
estimate an expected torque producible by the plurality of joints to satisfy a control input applied to the robotic arm, wherein the expected torque is based on a predefined dynamic model of the robotic arm; and
apply a torque limit to at least one of the plurality of joints that is based on and greater than the expected torque.

12. The surgical robot of claim 11, wherein the expected torque comprises a sum of a plurality of torques required to overcome gravitational loads applied to the robotic arm, frictional loads applied to the plurality of joints, and dynamic loads resulting from movement of one or more joints of the robotic arm.

13. The surgical robot of claim 11, wherein the expected torque includes an estimate of a model error caused by the predefined dynamic model upon which the expected torque is based.

14. The surgical robot of claim 11, wherein the expected torque includes an external interference parameter corresponding to an estimated drag applied to the robotic arm by objects external to the robotic arm.

15. The surgical robot of claim 11, wherein the expected torque comprises an initial expected torque, and wherein the processor is configured to determine a final expected torque that is bounded between an upper bound and a lower bound that is less than the upper bound.

16. The surgical robot of claim 15, wherein:
the upper bound comprises a sum of the initial expected torque and an external interference parameter corresponding to an estimated drag applied to the robotic arm by objects external to the robotic arm; and
the lower bound comprises a difference between the initial expected torque and the external interference parameter.

17. The surgical robot of claim 15, wherein:
the upper bound comprises a sum of the initial expected torque, an external interference parameter corresponding to an estimated drag applied to the robotic arm by objects external to the robotic arm, and an estimate of a model error caused by the predefined dynamic model upon which the expected torque is based; and
the lower bound comprises a difference between the initial expected torque and the sum of the external interference parameter and the model error.

18. A surgical robot, comprising:
a first end effector driven by a plurality of first joints of a first robotic arm of the surgical robot;
a second end effector driven by a plurality of second joints of a second robotic arm of the surgical robot; and
a processor communicatively coupled to the first robotic arm and the second robotic arm, the processor configured to:
apply a first torque limit to at least one of the plurality of first joints and/or at least one of the plurality of second joints in response to determining by the processor that the first robotic arm and/or the second robotic arm is actively moving; and
apply a second torque limit to at least one of the plurality of first joints and/or at least one of the plurality of second joints in response to determining by the processor that the first robotic arm and/or the second robotic arm is in a stationary state, wherein the second torque limit is different from the first torque limit.

19. The surgical robot of claim 18, wherein the processor is configured to determine the first torque limit using a dynamic model of at least one of the first robotic arm and the second robotic arm.

20. The surgical robot of claim 18, wherein the second torque limit is greater than the first torque limit.
